(19) United States
(12) Patent Application Publication
(10) Pub. No.: US 2006/0188558 A1
(43) Pub. Date: Aug. 24, 2006

(54) Transdermal systems having control delivery system

(75) Inventors: Kristin Jackson, Enosburg Falls, VT (US); Kenneth J. Miller II, St. Albans, VT (US); Pavan Bhat, Morgantown, WV (US)

Correspondence Address: LERNER, DAVID, LITTENBERG,;KRUMHOLZ & MENTLIK, 600 SOUTH AVENUE WEST, WESTFIELD, NJ, 07090, US

(73) Assignee: Mylan Technologies, Inc., St. Albans, VT (US)

(21) Appl. No.: 11/062,084

(22) Filed: Feb. 18, 2005

(51) Int. Cl. A61K 9/70

(52) U.S. Cl. 424/449

(57) ABSTRACT

Transdermal delivery systems are disclosed including a backing layer, a polymer membrane within the backing layer, an adhesive layer for attaching the delivery system to the patient's skin or mucosa, and a releasable layer covering the adhesive layer prior to use, the polymer membrane impregnated with sufficient fluid medium to alter the rate of transmission of an active agent through the polymer membrane, with the amount of fluid medium being greater than the amount retained by the fluid membrane upon drying.

### FIELD OF THE INVENTION

The present invention relates to transdermal delivery systems. More particularly, the present invention relates to transdermal delivery systems for delivering active agents to the skin or mucosa of a patient. Still more particularly, the present invention relates to such transdermal delivery systems in which a polymer membrane is utilized for controlling the rate of transmission of the active agent therethrough.

### BACKGROUND OF THE INVENTION

A considerable number of drug delivery devices are known in the art. These devices generally provide for a drug or other active agent to be released by diffusion from a reservoir or the like through the surface of the device to the skin or mucosa of a patient for the drug or other active agent. Most of the current transdermal systems can be divided into two major classes; that is, either reservoir systems or matrix-type systems. The reservoir systems generally comprise an enclosure of some kind filled with a fluid preparation of the active ingredient. In these systems, one side of the enclosure consists of a membrane which is permeable at least with respect to the active ingredient, and which is normally provided with a suitable adhesive. In the latter or matrix-type systems, the active ingredient is generally incorporated into a gel-type formulation or adhesive matrix, which is preferably also self-adhesive.

In connection with various membrane-containing devices, one objective has been to control the rate of administration, such as to delay the onset of therapeutic effect for significant time periods after application of the device. One such device, for example, is disclosed in Lee et al., U.S. Pat. No. 5,284,660. Like the others, this device employs one or more membranes between the agent reservoir and the surface where the agent is to be released. The membrane in this case is substantially free of undissolved agent, and is preferably formed from a material having low permeability in a first state and high permeability in a second state. The membranes are described as comprising various polymers which are hydrophilic or semi-hydrophilic, including polyvinyl alcohol, polyvinyl pyrrolidone, hydroxypropylcellulose, hydroxyethylcellulose, and hydroxypropylmethylcellulose.

In another prior art reference, Becher, U.S. Pat. No. 5,066,494, a multi-chamber system is used in order to attempt to obtain better control over transdermal drug application. Such a system is also said to help in preventing the flowing or dropping out of the complete active substance formulation therefrom.

In many devices, there is also a desire for an initial rapid drug administration or spike, and in the past this has been obtained, for example, by employing a contact adhesive layer in which the drug is contained. In references such as Reed, U.S. Pat. No. 4,877,618, a relatively constant but declining rate of administration is said to be obtained over extended time periods by employing a number of particulate-containing interlaminar layers which absorb the drug.

Another device is shown in Becher et al., U.S. Pat. No. 5,902,433, which in this case includes a number of chambers containing active substance, the chambers being in communication with each other, which are formed from top and bottom layers with an active-agent-containing substance therebetween, in which the top and bottom layers are brought together at predetermined sites to form channels or chambers thereof.

Another multi-compartment device is shown in Andriola et al., U.S. Pat. No. 4,666,441. In this case, the reservoir area is formed from a permeable porous membrane which is meant to control the rate of drug movement, and the membranes include polycarbonates, polyvinyl chlorides, polyamides, polysulfones, and the like.

Yet another patent disclosing a patch employing a membrane is Cavazza, U.S. Pat. No. 5,683,712. In this case, a microporous membrane is used to control the drug release through a subsequent adhesive layer and gel layers containing the drugs in question. No specific membrane materials are disclosed for this purpose in this patent.

Yet another transdermal product for application of active agents is disclosed in Kwiatek et al., U.S. Pat. No. 5,503,844. This patent discloses the use of cellular foam layers for use as carrier layers for active agents, with or without additional membrane layers. The foam layers disclosed in this patent are polyurethane foams, and conventional rate-controlling polymers are used therein.

A transdermal system which has already been marketed is the ESTRADERM® system marketed by Novartis, as shown schematically in FIG. 1 hereof. This system includes a patch product which includes an outer transparent polyester film 3, a drug reservoir 4 of estradiol and alcohol gelled with hydroxypropylcellulose, an ethylene vinyl acetate copolymer membrane 6, and an adhesive formulation 8 of light mineral oil and polyisobutylene. This is covered by a protective liner 10 of siliconized polyethylene terephthalate film for removal prior to use. The alcohol acts as a solvent or enhancing agent for movement of the drug through the skin. However, when the alcohol has been depleted, no driving force for the drug remains. As in the cases discussed above, the membrane utilized in this product is not only non-microporous, but does not have a capacity to retain a fluid composition, or an enhancing agent, therein.

There has thus developed a strong need for an improved patch system, and in particular one in which a membrane is employed which has a capacity to retain solvent and/or enhancing agents, such as volatile organic solvents, therein for the purpose of improving and controlling the delivery of the drug itself and improving the solubility of the drug in the patch systems utilized.

It is also true that in many of the present transdermal systems, since the systems including adhesive compositions must be subjected to rigorous drying conditions prior to use, that the drug itself also needs to be subjected to these conditions. Thus, systems have also been designed in which it does not prove necessary to subject the drug to such conditions and thereby improve its stability.

In many of the present systems, it is necessary that if a solvent is employed, it must at least be partially miscible with the adhesive systems utilized. Therefore, systems have also been sought in which one can employ solvents that are not fully compatible with the adhesive blend therein.

It is also apparent that in most of the prior transdermal systems the amount of solvent which can be retained in the patch itself is limited to the saturation concentration of the solvent in the adhesive itself. Therefore, systems have been sought which are not limited by this factor.

### SUMMARY OF THE INVENTION

In accordance with the present invention, these and other objects have now been realized by the discovery of a transdermal delivery system for delivering an active agent to the skin or mucosa of a patient comprising a backing layer, a polymer membrane disposed within the backing layer, an adhesive layer for attaching the transdermal delivery system to the skin or mucosa of the patient, and a releasable layer for covering the adhesive layer prior to attachment of the transdermal delivery system to the skin or mucosa of the patient, the polymer membrane impregnated with a predetermined amount of a fluid medium for altering the rate of transmission of the active agent through the skin or mucosa of the patient, the predetermined amount of the fluid medium being substantially greater than the amount of that fluid medium retained by the polymer membrane upon drying of the polymer membrane. Preferably, the fluid medium comprises a liquid solvent for the active agent, or an enhancer for the active agent, or an excipient (or solution of an excipient) for the active agent, or the active agent itself.

In accordance with another embodiment of the transdermal delivery system of the present invention, the adhesive layer comprises a first adhesive layer, and the device includes a second adhesive layer disposed between the backing layer and the polymer membrane.

In accordance with another embodiment of the transdermal delivery system of the present invention, the first and second adhesive layers comprise an adhesive matrix including one or more adhesives such as an acrylic, silicone, polyisoalkaline, rubber, vinyl acetate, polyisobutylene rubber, polybutadiene, styrene-butadiene, cellulose derivatives, polysaccharides, polyurethane elastomers, and polyester elastomers.

In accordance with another embodiment of the transdermal delivery system of the present invention, the fluid medium comprises a solvent for the active agent. In a preferred embodiment, the solvent comprises a C₂-C₈ alcohol.

In accordance with another embodiment of the transdermal delivery system of the present invention, the first adhesive layer includes the active agent. In another embodiment, the second adhesive layer includes the active agent. Preferably, both the first and second adhesive layers include the active agent.

In accordance with another embodiment of the transdermal delivery system of the present invention, the polymer membrane comprises a hydrophilic or hydrophobic polymer(s) or copolymer. In a preferred embodiment, the hydrophilic or hydrophobic polymer(s) or copolymer are selected from the following: polyolefin (e.g., polyethylene, polypropylene), ethylene vinyl acetate, polyvinyl acetate, polyether block amides, polyurethane, polyamides (e.g., nylon), cellulose and cellulose derivatives, polyvinyl chloride, polyvinyl alcohol, polystyrene, polymethyl methacrylate, polysilane, and polysiloxane.

In accordance with yet another embodiment of the transdermal delivery system of the present invention, the predetermined amount of the fluid medium comprises from about 0.5 to 10 mg/cm². In the preferred embodiment, the predetermined amount of the fluid medium comprises from about 1 to 7 mg/cm². Most preferably, the predetermined amount of the fluid medium comprises about 3.0 mg/cm².

In accordance with another embodiment of the transdermal delivery system of the present invention, the first and second adhesive layers comprise adhesives which are at least partially resistant to plasticization by a solvent for the active agent.

In accordance with one embodiment of the transdermal delivery system of the present invention, the system comprises a backing layer, a polymer membrane disposed within the backing layer, an adhesive layer for attaching the transdermal delivery system to the skin or mucosa of the patient, a releasable layer for covering the adhesive layer prior to attachment of the transdermal delivery system to the skin or mucosa of the patient, and a fluid medium distributed between the adhesive layer and the polymer membrane in a manner such that between about 2.5% and 100% of the fluid medium is disposed in the polymer membrane. Preferably, between about 50% and 100% of the fluid medium is disposed in the polymer membrane. In one embodiment, a greater proportion of the fluid medium is disposed in the polymer membrane than is disposed in the adhesive layer.

In accordance with the present invention, a method has also been discovered for manufacturing a transdermal delivery system for delivering an active agent to a patient in which the method comprises preparing an adhesive layer for attaching the transdermal delivery system to the skin or mucosa of the patient, providing a polymer membrane, impregnating the polymer membrane with a predetermined amount of a fluid medium for altering the rate of transmission of the active agent through the skin or mucosa of the patient, drying the adhesive layer without drying the polymer membrane, applying the adhesive layer to the impregnated polymer membrane, providing a backing layer and incorporating the adhesive layer and the polymer membrane into the backing layer, and providing a releasable liner adjacent to and protecting the adhesive layer prior to application of the transdermal delivery system to the skin or mucosa of the patient. In a preferred embodiment, the fluid medium comprises a solvent for the active agent, or an enhancer for the active agent, or an excipient (or solution of excipient) for the active agent, or the active agent itself.

In accordance with one embodiment of the method of the present invention, the adhesive layer comprises a first adhesive layer, and the method includes applying a second adhesive layer between the backing layer and the polymer membrane. In a preferred embodiment, the first and second adhesive layers comprise an adhesive matrix including an adhesive such as one of the following: acrylic, silicone, polyisoalkaline, rubber, vinyl acetate, polyisobutylene rubber, polybutadiene, styrene-butadiene, cellulose derivatives, polysaccharides, polyurethane elastomers and polyester elastomers.

In accordance with one embodiment of the method of the present invention, the fluid membrane comprises a solvent for the active agent. In a preferred embodiment, the solvent for the active agent comprises a C₂-C₁₈ alcohol.

In accordance with another embodiment of the method of the present invention, the method includes adding the active agent to the first adhesive layer. In another embodiment, the method includes adding the active agent to the second adhesive layer. In a preferred embodiment, the method includes adding the active agent to both the first and second adhesive layers.

In accordance with another embodiment of the method of the present invention, the polymer membrane comprises a polymer such as either a hydrophilic or hydrophobic polymer. Preferably, the hydrophilic polymer is a polyolefin (e.g., polyethylene) or polyethylene/ethylene vinyl acetate copolymer.

In accordance with another embodiment of the method of the present invention, the predetermined amount of the fluid medium comprises from 0.5 to 10 mg/cm², preferably from about 1 to 7 mg/cm², and most preferably 3.0 mg/cm².

In accordance with another embodiment of the method of the present invention, the first and second adhesive layers comprise adhesives which are at least partially resistant to plasticization by the solvent for the active agent.

### BRIEF DESCRIPTION OF THE DRAWINGS

The present invention may be more fully understood with reference to the following detailed description which, in turn, refers to the figures, in which:

FIG. 1 is a side, elevational, enlarged, cross-sectional view of a liquid reservoir transdermal device in accordance with the prior art; and

FIG. 2 is a side, elevational, enlarged, cross-sectional view of a transdermal device in accordance with the present invention;

### DETAILED DESCRIPTION

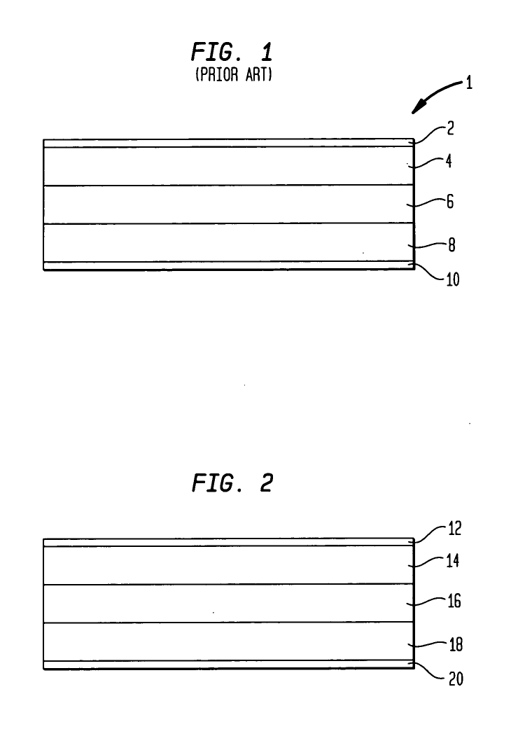

Referring first to FIG. 1, a transdermal patch product of the prior art is shown therein. The prior art product is intended to represent known products, such as the ESTRADERM® transdermal patch product sold by Novartis. This patch product includes a backing layer 2 which is impermeable to fluids contained within the patch, and a reservoir 4 comprising a drug or active agent along with an alcohol-containing fluid gel reservoir composition. A conventional microporous membrane 6 is then included along with an adhesive layer also containing active agent or drug therein. The patch is covered by a releasable liner 10 which is removed prior to application of the adhesive layer 8 to the skin or mucosa of the patient. Use of alcohol-like substances acts as an enhancer since these small molecule compositions can travel through the skin far faster than the drug components used in these products. The alcohol material thus initially carries additional amount of the drug (estradiol in this case) through the skin, and the patient receives an initial “spike” of drug composition. However, as soon as the alcohol is depleted, little driving force remains for the estradiol to pass through the skin of the patient. Attempts have thus been made to obtain equivalent “spikes” of the drug composition without using materials such as alcohol. These attempts have not proven to be successful, however.

Referring to FIG. 2, a comparable transdermal patch product of the present invention is shown, including backing layer 12; first adhesive layer 14, preferably containing active agent therein; membrane 16; a second adhesive layer 18, again preferably containing active agent therein; and releasable liner 21 thereon.

In a preferred composition as shown in FIG. 2, in addition to the polymer membrane 16, either one of adhesive layers 14 or 18, or both, are preferably employed. Furthermore, even when two adhesive layers 14 and 18 are used, they can constitute different adhesive formulations, and one or the other or both of these adhesive layers 14 and 18 can include the active agent itself dispersed therein. As a separate matter, the polymer membrane 16 can include active agent dissolved in the solvent contained within the pores of the membrane. This, of course, can also be the only source of active agent, or active agent can in addition be included in one or both of adhesive layers 14 and 18. These various combinations permit one to custom make the specific transdermal patch system so that an appropriate drug and drug application regimen can be provided. This can include an initial “spike” in the drug delivery, it can include a shortening of the lag time between application of the patch and drug delivery, and it can alter the solubility of the drug in the system or constituent layers for such purposes.

The polymer membrane 16 is an essential element of the present invention, since it must have sufficient capacity (via pores or as a result of swelling) so as to retain solvent or other fluid media within the membrane. Thus, the presence of such volatile organic solvents in the membrane of the present invention helps improve the delivery of the drug by acting as an enhancer and/or by improving the solubility of the drug in the patch itself. Thus, while volatile solvents were used in the past, much of the solvents were driven off during drying of the patches prior to use. In accordance with this invention, however, it is possible to thoroughly dry the adhesive matrix without driving off the solvent which is desired, by incorporating the solvent into the membrane in accordance with this invention, preferably subsequent to the drying process.

There are thus at least two elements which combine to provide the present invention. First, there is the incorporation of a fluid medium into the membrane itself. Second, there is the drying of the adhesive layer or layers, or indeed all of the patch except for the membrane, prior to completion of the patch; i.e., separately from the membrane. Thus, the membrane itself is not subjected to the drying conditions; i.e., elevated temperatures, which are applied to the adhesive layer(s)themselves. It is in this manner that it now becomes possible to incorporate into these transdermal systems far greater amounts of these fluid media than has previously been the case.

The advantages now achievable by means of this invention include the ability to more accurately control the overall drug application process, such as by applying or extending the period of a drug “spike” and/or the entire period of drug application. It is also now possible to utilize larger amounts of fluid media which would normally result in reduction, or even elimination, of the adhesive character of the adhesive layer(s) if applied thereto. In addition, it is also possible to utilize a fluid medium which is entirely incompatible with the adhesive layer(s), since the fluid does not need to be carried by that adhesive layer(s), but can be retained by the polymer membrane. All of this permits a radical alteration in the overall design of these transdermal drug delivery systems.

There are many transdermal systems which are known to employ membranes, primarily for rate-controlling purposes, or as a component in a gel reservoir system such as that set forth above. However, the membranes of the present invention, employed in the specific systems hereof, perform an entirely different function; namely, of retaining solvent, enhancer, excipient and/or drug compositions for the purpose of controlling the application of the drug, modifying the rate of drug delivery, or selectively modifying the solubility of the drug in the system.

The membranes usable in accordance with the present invention possess sufficient capacity to retain within the membrane greater than 5 mg/10 cm² of solvents such as short chain alcohols (2 to 18 carbon atoms, preferably ethyl alcohol) preferably from 5 to 100 mg/10 cm², and preferably at least about 30 mg/10 cm².

The polymer membranes useful in accordance with the present invention can include a variety of both hydrophilic and hydrophobic polymers or copolymers. These polymers and copolymers can include polyolefins, such as polyethylene and polypropylene, ethylene-vinyl acetate, polyvinyl acetate, polyether block amides, polyurethane, polyamides, such as nylon, cellulose and cellulose derivatives, polyvinyl chloride, polyvinyl alcohol, polystyrene, polymethyl methacrylate, polysilane, and polysiloxane. Among the preferred membranes for use in the present invention are included ethylene-vinyl acetate, polyethylene, such as CoTran®9711 of 3M Corporation, and SULOPOR® of DSM Corporation, or ultra-high molecular weight polyethylene membrane.

Other examples of the polymer membranes which can be used in accordance with this invention include copolyester membranes, preferably between 0.5 and 3.0 mils in thickness, with MVTRs between about 1,000 and 15,000 g/m²/24 hrs.; polyurethane membranes which are from about 0.5 to 3.0 mils in thickness with MVTR values between about 1,000 and 10,000 g/m²/24 hrs.; polyether block amides with MVTRs between about 2,000 and 10,000 g/m²/24 hrs.; 9% ethylene vinyl acetate polymer membranes which are between about 1 and 5 mils in thickness; and hydrophilic polyethylene membranes which are between about 1.0 and 10.0 mils in thickness.

The various pharmaceutically active agents which can be used in accordance with the present invention are legion. Indeed, essentially any active agent which has been or could be applied transdermally is a candidate for the present invention. With any such active agent, or drug, or class thereof, the present invention can be utilized to manipulate the natural rate of transdermal or transmucosal delivery, and/or to insert a greater amount of the active agent into a dosage form thereof. Manipulation of the natural rate of delivery could, for example, be in connection with certain steroids. That is, endogenous steroid levels, such as with estrogens, progestens and androgens, follow circadian patterns. The present invention could then be employed to obtain a “spike” shortly after application. Other active agents, such as analgesics can sometimes work fast by delivering a bolus in order to terminate pain, followed by a steady but slower rate of delivery to prevent return of the pain.

On the other hand, insertion of greater amounts of active agent into a dosage form could be applied, for example, in connection with active agents such as testosterone. This drug can be suspended in an acrylic adhesive in order to delivery a therapeutic dose, but the testosterone can crystallize within the matrix in an unpredictable manner. By application of the present invention, delivery of the drug can be accomplished with sufficient drug loaded into the system, and there would be no driving force for crystallization of the testosterone, unless the solvent being utilized were lost from the membrane during storage. In another example, attempts to load a sufficient amount of fentanyl into a polyisobutylene adhesive matrix in a patch in order to deliver a therapeutic dose thereof over three days will result in the adhesive matrix having lost its adhesive properties. By utilizing the present invention, however, the fentanyl will remain dissolved throughout the adhesive matrix and that matrix can still remain tacky

As for the active agents themselves, again as noted above, there is essentially no limit on the potential use of any such active agents which can be delivered transdermally or transmucosally.

Suitable systemic drugs include, without limitation, anti-microbial agents such as penicillin, tetracycline, oxytetracycline, chlortetracycline, chloramphenicol, and sulfonamides; sedatives and hypnotics such as pentabarbital sodium, phenobarbital, secobarbital sodium, codeine, (a-bromoisovaleryl)urea, carbromal, and sodium phenobarbital; psychis energizers such as a 30(2-aminopropyl) indole acetate and 3-(2-aminobutyl) indole acetate; tranquilizers such as reserpine, chlorpromazine hydrochloride, and thiopropazate hydrochloride; hormones such as adrenocorticosteroids, for example, 6α-methylprednisolone; androgenic steroids, for example, methyltestosterone, and fluoxymesterone; estrogenic steroids, for example estrone, 17β-estradiol and ethinyl estradiol; progesterone, and norethindrone; and thyroxide; antipyretics such as aspirin, salicylamide, and sodium salicylate; morphine and other narcotic analgesics; anti-diabetics, e.g., insulin; cardiovascular agents, e.g. nitroglycerin, and cardiac glycosides such as digitoxin, digoxin, ouabain; anti-spasmodics such as atropine, methscopolamine bromide, methscopolamine bromide with phenobarbital; anti-malarials such as the 4-aminoquinolines, 9-amino-quinolines, and pyrimethamine; and nutritional agents such as vitamins, essential amino acids, and essential fats.

The backing layer which is impermeable to the drug, and the adhesive primarily has as its objectives to prevent seepage of the active agent or adhesive through the backing layer. If, for example, the backing layer is coated on the surface in contact with the remainder of the device with an adhesive layer that is active agent impermeable, this impermeable adhesive layer will essentially perform this purpose even if the backing layer is not totally impermeable to the active agent. Thus, it is not necessary in all instances that the backing layer be impermeable to the active agent, although in most instances it normally is, and when it is not a layer providing this barrier function, such as an active-ingredient-impermeable adhesive layer, it will be situated between the backing layer and the remainder of the device such as the membrane.

The backing layer may also be impermeable to the solvent or other fluid medium contained within the transdermal system. However, in some embodiments it is possible that the backing layer might be permeable to the solvent or other fluid component therein. For example, a portion of this liquid medium might be permitted to evaporate through the backing layer. This could thus provide a cooling effect, or it could act as a secondary method for attenuating the “spike” of active agent through the skin or mucosa of the patient by allowing a portion of the solvent or the enhancer to escape from the system other than by passing through the skin or mucosa of the patient.

The actual material used for the outer surface of the backing layer will depend on the properties of the materials in contact therewith. Some suitable materials include, for example, cellophane, cellulose acetate, ethyl cellulose, plasticized vinyl acetate-vinyl chloride copolymers, ethylene-vinyl acetate copolymer, polyethylene terephthalate, nylon, polyethylene, polypropylene, polyvinylidine chloride (e.g., SARAN), paper, cloth and aluminum foil. The material used is preferably impermeable to the active gent. The material which forms this backing layer may be flexible or non-flexible. Preferably, a flexible backing layer is employed to conform to the shape of the body to which the device is attached.

Preferably, the material which forms the backing layer, such as layer 12, is a film or a composite film. The composite can be a metallized (e.g., aluminized) film or a laminate of two or more films or a combination thereof. For example, a laminate of polyethylene terephthalate and polyethylene or a polyethylene/metallized polyethylene terephthalate/polyethylene laminate can be employed. The preferred polymers include polyethylene, polypropylene, polyvinyl chloride, polyesters such a polyethylene terephthalate (MYLAR), and polyvinylidine chloride (SARAN). More particularly, a highly preferred composition of the present invention employs highly occlusive layers of polyethylene terephthalates or polyvinylidine chloride as a backing layer.

The transdermal patch systems of the present invention also include a release or releasable layer for temporarily covering the adhesive surface prior to application. The release layer can be made of the same material suitable for use in the backing layer as discussed above. Such materials are preferably made releasable from the adhesive layer by, for example, conventional treatment with silicone, TEFLON, or other suitable coating on the surface thereof. The removal of the device from the release layer may also be provided by mechanical treatment of the release layer, such as by embossing same.

The release layer can also comprise various layers including paper or paper-containing layers or laminates; various thermoplastics, such as extruded polyolefins, such as polyethylene; various polyester films, foil liners, other such layers, including fabric layers, coated or laminated to various polymers, as well as extruded polyethylene, polyethylene terephthalate, various polyamides, and the like, with the polyester films being preferred. The release layer can also comprise vacuum metallized films such as metallized polyester or polypropylene formed by vacuum deposition of aluminum for UV and oxygen resistance.

Another possible release layer of the present invention includes a laminate of an outer foil layer and an inner layer of plastic, such as polyethylene or the like, which is rendered releasable not only by means of a siliconized coating, but which also includes an embossed or roughened surface. Embossment of the surface can be accomplished by a number of conventional methods. In general, preparation of embossed surfacing can be accomplished by the use of male-female tooling, preferably enhanced by the application of heat. The principal intention of this embossment process is to roughen the surface or render it uneven so that less than the entire surface will be in physical contact with the corresponding adhesive layer.

The preferred release layers of the present invention include polyester films, preferably including a siliconized or fluorocarbon coating thereon, such as SCOTCH PAK 1022 from 3M Corporation.

As is also discussed above, the fluid medium which is incorporated into the membranes of the present invention can include water, C₁-C₃ alcohols, dimethyl sulfoxide, N,N-dimethylacetamide, polyethylene glycol, polysorbitols, polyethylene oxide, polyoxyethylene, dimethicone, mineral oil/paraffin, vegetable oils, and the like.

In accordance with the present invention, the solvent to be incorporated into the membrane is preferably an alcohol. Alcohols in accordance with the present invention can include monoalcohols, such as methanol, ethanol, propanol, isopropanol, butanol, and tertbutyl alcohol. The alcohol may also be a generally low molecular weight diol, triol, or polyol, i.e., glycols such as propylene glycol, triols such as glycerol, and polyalkylene glycol having an average molecular weight of less than about 400. For example, the solvent may be polyethylene glycol having an average molecular weight of between about 200 and about 400. The solvent in accordance with the present invention can thus comprise a normal short chain polyol of between about 2 and about 4 carbons in length. Such polyols may include 1,4 butanediol, glycerol, ethylene glycol, propylene glycol, and the like. Also useful in accordance with the present invention are acetates such as, for example, ethyl acetate, cellulose acetate, vinyl acetate and the like.

It is also possible to use additional components in the transdermal systems of the present invention, such as dyes, permeation enhancers, cross-linkers, adhesion promoters, gelling agents, crystallization inhibitors, anti-inflammatory agents, and the like.

Penetration enhancers can also be included as the fluid media of the present invention. These penetration enhancers are intended to promote penetration of the active agent through the skin, and suitable enhancers include those described in U.S. Pat. No. 5,503,844, including monovalent, saturated and unsaturated aliphatic and cycloaliphatic alcohols having 6 to 12 carbon atoms such as cyclohexanol, lauryl alcohol, and the like; aliphatic and cycloaliphatic hydrocarbons such as mineral oil; cycloaliphatic and aromatic aldehydes and ketones such as cyclohexanone; N,N-di(lower alkyl)acetamides such as N,N-diethyl acetamide and N,N-dimethyl acetamide, N,N-dimethyl acetamide, N-(2-hydroxyethyl)acetamide and the like; aliphatic and cycloaliphatic esters such as isopropyl myristate and lauricidin; N,N-di(lower alkyl) sulfoxides such as decylmethyl sulfoxide; essential oils, nitrated aliphatics, aliphatic and cycloaliphatic hydrocarbons such as N-methyl-2-pyrrolidone and azone; salicylates, polyalkylene glycol silicates; aliphatic acids such as oleic acid and lauric acid, terpines such as cineole, surfactants such as sodium lauryl sulfate, siloxanes such as hexamethyl siloxane; mixtures of the above materials; and the like.

Examples of fluid carriers that may be combined with the active agent in the membrane layer include simple alcohols, polyethylene glycols, polypropylene glycols, polyester and polyether polyols, epoxidized linseed oils and simple liquid esters such as triethyl citrate, dicyclohexyl phthalate, diisoacyl adipate, fatty acids (oleic, lauric and the like), salts of fatty acids, fatty alcohols, fatty esters (CERAPHYLS and the like), terpenes and like. The preferred fluid carriers include short-chain alcohols, fatty acids, fatty esters, fatty alcohols, polyethylene glycols and polypropylene glycols.

Examples of binders that can be combined with the active agent in the adhesive and/or membrane layers of the present invention include conventional hydrogels formed using water-soluble or water-insoluble gums or resins, with or without known cross-linking agents. The gums or resins include agarose, alginates, alkyl and hydroxyalkyl celluloses, such as hydroxyethyl cellulose and hydroxypropyl cellulose, amylopectin, arabinogalactin, carboxymethyl cellulose, carrageenan, eucheuma, ucoidan, furcellaran, gelatin, guar gum, gum agar, gum arabic, gum ghatti, gum karaya, gum tragacanth, pypenia, keratin laminaran, locust bean gum, pectin, polyacrylamide, poly(acrylic)acid and homologs, polyethylene glycol, poly(ethylene oxide), poly(hydroxyalkyl) methacrylate, polyvinyl alcohol, polyvinylpyrrolidone, propylene glycol alginate, starch and modified analogs, tamarind gum, N-vinyl lactam polysaccharides and xanthan gum. In addition, such hydrogels can be formed by the copolymerization and cross-linking of both hydrophilic and hydrophobic monomers, such as hydroxy-alkyl esters of acrylic acid and methacrylamide, n-vinyl-1-pyrrolidone, alkyl acrylates and methacrylates, vinyl acetate, acrylonitrile and styrene. Other binders suitable for use with the present invention include veegum, higher molecular weight polyglycols, and the like.

The binders that are preferred for use with the present invention include cellulose esters, polyvinyl pyrrolidones and polyacrylates. Binders in accordance with the present invention can be prepared as a liquid, paste, semi-solid or solid that is combined with the active agent and incorporated into the membrane layer.

A therapeutic adhesive formulation for use in accordance with the present invention includes various adhesive formulations which can be used as part of the transdermal drug delivery systems hereof. Preferably, these adhesive formulations are monolithic structures and preferably include both an adhesive formulation and a pharmaceutically active agent therein. The adhesive formulations which can be used in accordance with the present invention include many such formulations known in the art. Broadly these include acrylics, silicones, polyisoalkalines, rubbers, vinyl acetates, polyisobutylene rubber, polybutyldiene, styrene-butadiene(or isoprene)-styrene block copolymer rubber, acrylic rubber, and natural rubber; vinyl-based high molecular weight materials such as polyvinyl alkyl ether, polyvinyl acetate, a partially saponified product of polyvinyl acetate, polyvinyl alcohol and polyvinyl pyrrolidone; cellulose derivatives such as methyl cellulose, carboxylmethyl cellulose and hydroxypropyl cellulose; polysaccharides such as pullulan, dextrin and agar; polyurethane elastomers; and polyester elastomers. Of course, the adhesives must be biocompatible and nonirritating. They must also allow for a patch to adhere firmly to the skin or mucosa of a patient in need of treatment by a patch, but not be so adhesive so as to injure the patient as the patch is removed. It is also important that the adhesive be selected such that it is compatible with the other components of the therapeutic adhesive formulation of the present invention. It has been found that, as a group, the acrylic adhesives are particularly useful and compatible in this regard and therefore, it is preferred that the adhesive used be acrylic based. More specifically, acrylic adhesives in accordance with the present invention may preferably be (meth)acrylic acid such as butyl (meth)acrylate, pentyl (meth)acrylate, hexyl (meth)acrylate, heptyl (meth)acrylate, octyl (meth)acrylate, nonyl (meth)acrylate, decyl (meth)acrylate, undecyl (meth)acrylate, dodecyl (meth)acrylate, and tridecyl (meth)acrylate, and copolymers of at least one of the above esters and other monomers copolymerizable therewith.

Examples of the preferred polyacrylate adhesives for use in the transdermal system of the present invention are those sold under the trademark DuroTak®87-2194, 87-2620, 87-2052, 87-2852, 87-2054, 87-2979 and 87-6173 by National Starch and Chemical Corporation. Other suitable adhesives are sold under the trademark GELVA-Multipolymer Solution, GELVA 2873 and 2883 by Surface Specialties, Inc.; and silicone adhesives sold under the trademark BIO-PSA 7-4300 and 7-4500 by Dow Corning Corporation. Other preferred adhesives include polyisobutylene and styrene-butadiene rubber adhesives.

Examples of the copolymerizable monomer include carboxyl group-containing monomers such as (meth)acrylic acid, itaconic acid, crotonic acid, maleic acid, maleic anhydride and fumaric acid; sulfoxyl group-containing monomers such as styrenesulfonic acid, arylsulfonic acid, sulfopropyl acrylate, (meth)acryloyloxynaphthalenesulfonic acid, acrylamidomethylpropanesulfonic acid and acryloyloxybenzenesulfonic acid; hydroxyl group-containing monomers such as hydroxyethyl (meth)acrylate and hydroxypropyl (meth)acrylate; amide group-containing acrylic monomers such as (meth)acrylamide, dimethyl(meth)acrylamide, N-butylacrylamide, tetramethylbutylacrylamide and N-methylol(meth)acrylamide; alkylaminoalkyl group-containing acrylic monomers such as aminoethyl (meth)acrylate, dimethylaminoethyl (meth)acrylate, diethylaminoethyl (meth)acrylate and tertbutyl (meth)acrylate; alkyl esters of acrylic acid containing an ether bond in the molecule thereof such as methoxyethyl (meth)acrylate, ethoxyethyl (meth)acrylate, butoxyethyl (meth)acrylate, tetrahydrofurfuryl (meth)acrylate, methoxyethylene glycol (meth)acrylate, methoxydiethylene glycol (meth)acrylate, methoxypolyethylene glycol (meth)acrylate and methoxypolypropylene glycol (meth)acrylate; vinyl monomers such as N-(meth)acryloylamino acid; functional monomers such as urethane, urea or isocyanate ester of acrylic acid; and vinyl monomers such as (meth)acrylonitrile, vinyl acetate, vinyl propionate, vinyl pyrrolidone, vinyl pyridine, vinyl pyrazine, vinyl piperadine, vinyl piperidone, vinyl pyrimidine, vinyl pyrrole, vinyl imidazole, vinyl caprolactam, vinyl oxazole, vinyl thiazole, vinyl morpholine, styrene, a-methylstyrene and bis(N,N′-dimethylaminoethyl) maleate.

The above alkyl esters of (meth)acrylic acid and copolymerizable monomers include isomers in which the alkyl portion is straight or branched, and isomers and derivatives in which the position of substituents is different.

It is desirable from a standpoint of the balance between adhesive properties to the skin and cohesion that the ratio of the alkyl ester of (meth)acrylic acid to the copolymerizable monomer in the acrylic pressure-sensitive adhesive material is 50:50 to 99:1 by weight. When alkyl esters of (meth)acrylic acid containing an ether bond in the molecule thereof are used from the standpoint of the low skin irritating properties, it is desirable that the ratio of the alkyl ester of (meth)acrylic acid/the alkyl ester of (meth)acrylic and containing an ether bond in the molecule/the other copolymerizable monomer is 40 to 80/59 to 10/1 to 40.

It is preferred that the adhesive formulations be subjected to suitable chemical cross-linking treatment (e.g., copolymerization of cross-linkable monomers and addition of a cross-linking agent) or physical cross-linking treatment (e.g., irradiation with ultraviolet rays and ionizing radiations such as electron beam).

In accordance with the present invention, the amount of adhesive generally utilized ranges from between about 30 to about 99 percent by weight based on the weight of the resulting formulation (excluding backing and release films). Preferably, the amount of adhesive used ranges from between about 65 to about 95 percent by weight based on the total weight of the formulation (excluding backing and release films).

Acrylic polymeric adhesives in accordance with this aspect of the present invention include between about 40% and about 90% of a C₄-C₁₂ alkyl acrylate as the principal monomeric component. Any alkyl acrylate having between 4 and 12 carbons which has been used for the formulation of transdermal adhesives can be used, although, of course, other acrylates are also contemplated. Traditional C₄-C₁₂ alkyl acrylates useful in accordance with the present invention include, for example, 2-ethylhexyl acrylate, butyl acrylate, n-decyl, n-nonyl, 2 ethyoctyl, isooctyl and dodecyl-acrylate Generally, the C₄-C₁₂ alkyl acrylate in accordance with the present invention will be used in a matter of between about 40 and about 90% based on the weight of the finished adhesive material. More preferably, however, the amount of the C₄-C₁₂ alkyl acrylate will range from between about 60% to about 80% by weight, based on the weight of the adhesive.

The properties of the acrylic polymeric adhesive can be dramatically altered depending upon whether or not a hardening monomer is used and the type of hardening monomer used. It has been found that the use of between about 10% and about 40% by weight of a C₁-C₄ alkyl acrylate hardening monomer, in combination with the C₄-C₁₂ alkyl acrylate, can provide an acrylic polymeric adhesive system capable of providing desirable therapeutic delivery, as well as structural integrity. Examples of C₁-C₄ alkyl acrylate hardening monomers useful in accordance with the present invention include methyl acrylate, methyl methacrylate, ethylacrylate, ethyl methacrylate, hydroxyethyl acrylate and hydroxy propyl methacrylate. More preferably, the amount of C₁-C₄ alkyl acrylate hardening monomer useful in accordance with the present invention ranges from between about 15% to about 30% based on the weight of the adhesive.

In accordance with the present invention, it is also desirable to use a functionalizing monomer which facilitates cross-linking. Functionalizing monomers provide functional groups for cross-linking. Such functionalizing monomers are well known in the art and include, for example, acrylic acid, hydroxy ethylacrylate, methacrylic acid, and acrylamide. It should be noted, however, that when using an acrylate hardening monomer in an acid form, it is preferred to use a functionalizing monomer, such as acrylic acid, whereas, where the hardening monomer is an alcohol, compounds such as hydroxy methylacrylate should be chosen. Functionalizing monomers are generally provided in the range of about 1% to about 20%.

It is also desirable to include a cross-linking agent. Cross-linking agents can include butyl titanate, polybutyl titanate, aluminum zinc acetate and other multivalent metals, methylol ureas and melamines Generally the cross-linking agent is provided in an amount of between about 0.005 and about 2% the adhesive.

Cross-linking can be effected in many ways depending upon a number of factors. Most importantly, cross-linking depends upon the mode of action of the cross-linking agent. Most of the acrylic polymeric adhesive formulations commercially available use cross-linking agents which will be activated upon the drying of the formulation. It is not the heat which activates these agents but rather the removal of the solvent by, for example, evaporation or drying. Drying to remove these solvents can by done under completely conventional conditions such as 100 to 140° F. It should be noted that certain formulations are commercially available without cross-linkers. For example, GELVA 1430 is identical to GELVA 1753 except that it does not include a cross-linker. This allows one to accommodate situations where no cross-linking is needed (such as when very low concentrations of drug are used) or to custom select a cross-linker that has a different mode of action.

The present invention can also be appreciated with reference to the following examples.

### EXAMPLE 1

An active estradiol blend was made by sonicating estradiol hemihydrate and 200 proof ethyl alcohol in a 4-ounce glass jar. Polyvinyl pyrrolidone, fumed silica, propylene glycol and DuroTak®87-2194 adhesive were added and the blend was mixed using an air driven mixing blade. The active blend was coated knife over a roll as the skin contact layer at 18 mil wet on Medirelease®2226, then dried at 54° C. for 5 minutes and 85° C. for 8 minutes. The anchor layer was placebo polyisobutylene coated at 8 mil wet on Medirelease®2226, dried at 54° C. for 5 minutes and 85° C. for 8 minutes, then laminated to 0.5 mil PET. The release liner was peeled off the polyisobutylene anchor layer. A piece of CoTran®9711, the same size as the anchor adhesive, was placed on the exposed anchor adhesive. The anchor layer with the CoTran®9711 was soaked in a bath of 200 proof ethyl alcohol for 2 minutes. The anchor layer was removed from the bath. The active skin contact layer was then laminated on top of the exposed membrane impregnated with ethyl alcohol. Patches were die cut and pouched in Barex pouching. The patch exhibited a delivery “spike” in vitro.

### EXAMPLE 2

An active adhesive blend was made by sonicating estradiol hemihydrate and ethanol until the estradiol hemihydrate was dispersed (approx. 3 min.). Polyvinyl pyrrolidone was added to the premix and dissolved. Next, propylene glycol was added and swirled. Finally, DuroTak®87-2194 adhesive and fumed silica were added. The blend was mixed in a glass jar with an air driven mixing blade, after which the blend was rolled on a jar roller overnight to degas. An active adhesive blend was coated knife over roll twice on Medirelease®2226. An anchor layer was coated at 8 mil wet, and the skin contact layer was coated at 19 mil wet. Both layers were dried at 41° C. for 4 minutes and 77° C. for 4 minutes. To make the finished patch, the anchor layer was laminated to Mediflex®1000. The release liner was peeled off the anchor layer. A 9% ethylene vinyl acetate membrane (2.0 mil) was saturated with 200 proof ethyl alcohol by submerging the membrane in an ethyl alcohol bath. The saturated membrane was removed from the bath and placed on the adhesive side of the anchor layer. The skin contact layer (adhesive side) was laminated on top of the exposed membrane with ethanol. This formulation exhibits a delivery “spike” in vitro.

### EXAMPLE 3

An active blend was made by sonicating the estradiol hemihydrate (0.41 g) in 200 proof ethyl alcohol (3.09 g) for 3 minutes. The polyvinyl pyrrolidone (1.04 g) was dissolved in the premix and sonicated for 3 minutes. Propylene glycol (4.80 g), DuroTak®87-2194 (60.79 G) and fumed silica (0.17 g) were added to the premix. The entire blend was mixed with an air driven mixing blade for 3 minutes, after which a glass jar was rolled on the jar roller overnight to degas. The laminates were coated knife over roll at 14 mil wet on Medirelease®2249 to obtain a coat weight of 55.0 g/m². the laminate was dried at 41° C. for 4 minutes and 77° C. for 4 minutes. The first laminate was laminated to Mediflex®1500 backing as the anchor layer. The release liner was peeled off the anchor layer. A piece of DSM Solupor 10P05A membrane was submerged in a bath of 200 proof ethyl alcohol for at least 1 minute. The membrane was removed form the bath and wiped with a lint-free wipe to remove excess ethyl alcohol. After 1 minute in ambient air, the wet membrane was placed on the exposed adhesive of the anchor layer. The second laminate (skin contact layer) was immediately laminated on top of the exposed membrane loaded with ethyl alcohol. Appropriate size patches were immediately die cut from the laminate and sealed in polyethylene pouching material. This formulation exhibits a delivery “spike” in vitro.

### EXAMPLE 4

An active blend was made by sonicating albuterol sulfate in ethyl acetate for 5 minutes in a 4-ounce glass jar. Mineral oil, lauryl alcohol, and lauric acid were added to the premix. Polyisobutylene adhesive was added to the jar and the blend was mixed with an air driven mixing blade for 3 minutes, after which the blend was rolled on the jar roller to degas. The blend was coated twice on Medirelease®2226 at 10 mil wet, and dried at 55° C. for 5 minutes and 85° C. for 8 minutes. One laminate was laminated to the Mediflex@1000 backing, the release liner was peeled off and 7 cm² pieces of CoTran®9711 were placed on the exposed adhesive. Fifty μL of 1-Octanol was pipeted onto each piece of CoTran®9711. The other laminate was laminated on top of the CoTran®9711 impregnated with 1-Octanol and 10 cm² patches were die cut. The patches were pouched immediately. The in vitro flux lag time was shortened compared to control without a membrane impregnated with 1-Octonal.

### EXAMPLE 5

An active blend was made by sonicating albuterol sulfate in ethyl acetate for 3 minutes in a 4-ounce glass jar. Mineral oil and lauric acid were added to the premix. Polyisobutylene adhesive was added to the jar and the blend was mixed with an air-driven mixing blade for 3 minutes, after which the blend was rolled on the jar roller to degas. The blend was coated twice on Medirelease®2226 at 50 g/m², and dried at 55° C. for 5 minutes and 85° C. for 8 minutes. One laminate was laminated to the Mediflex®1000 backing, the release liner was peeled off and 10 cm² pieces of CoTran®9711 impregnated with lauryl alcohol were placed on the exposed adhesive. The second laminate was laminated on top of the CoTran®9711 impregnated with lauryl alcohol and 10 cm² patches were die cut.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A transdermal delivery system for delivering an active agent to the skin or mucosa of a patient comprising a backing layer, a polymer membrane disposed within said backing layer, an adhesive layer for attaching said transdermal delivery system to said skin or mucosa of said patient, and a releasable layer for covering said adhesive layer prior to attachment of said transdermal delivery system to said skin or mucosa of said patient, said polymer membrane impregnated with a predetermined amount of a fluid medium for altering the rate of transmission of said active agent through said polymer membrane to said skin or mucosa of said patient, said predetermined amount of said fluid medium being substantially greater than the amount of said fluid medium retained by said polymer membrane upon drying of said polymer membrane.

2. The transdermal delivery system of claim 1 wherein said fluid medium comprises a liquid selected from the group consisting of a solvent for said active agent, an enhancer for said active agent, an excipient for said active agent, and said active agent.

3. The transdermal delivery system of claim 1 wherein said adhesive layer comprises a first adhesive layer, and including a second adhesive layer disposed between said backing layer and said polymer membrane.

4. The transdermal delivery system of claim 3 wherein each of said first and second adhesive layers comprise an adhesive matrix including an adhesive selected from the group consisting of acrylic, silicone, polyisoalkalines, rubber, vinyl acetate, polyisobutylene rubber, polybutadiene, styrene-butadiene, cellulose derivatives, polysaccharides, polyurethane elastomers and polyester elastomers.

5. The transdermal delivery system of claim 4 wherein said adhesive is selected from the group consisting of acrylics, silicone, rubber, styrene-butadiene rubber, and polyisobutylene.

6. The transdermal delivery system of claim 5 wherein said adhesive comprises an acrylic adhesive.

7. The transdermal delivery system of claim 2 wherein said fluid medium comprises a solvent for said active agent.

8. The transdermal delivery system of claim 7 wherein said solvent comprises a C₂-C₈ alcohol.

9. The transdermal delivery system of claim 4 wherein said first adhesive layer includes said active agent.

10. The transdermal delivery system of claim 4 wherein said second adhesive layer includes said active agent.

11. The transdermal delivery system of claim 4 wherein both said first and second adhesive layers include said active agent.

12. The transdermal delivery system of claim 1 wherein said polymer membrane comprises a polymer selected from the group consisting of hydrophilic and hydrophobic polymers and copolymers.

13. The transdermal delivery system of claim 12 wherein said polymer membrane is selected from the group consisting of polyolefins, ethylene-vinyl acetate, polyvinyl acetate, polyether block amides, polyurethane, polyamides, cellulose, cellulose derivatives, polyvinyl chloride, polyvinyl alcohol, polystyrene, polymethyl methacrylate, polysilane and polysiloxane.

14. The transdermal delivery system of claim 1 wherein said predetermined amount of said fluid medium comprises from about 0.5 to 10 mg/cm².

15. The transdermal delivery system of claim 14 wherein said predetermined amount of said fluid medium comprises from about 1 to 7 mg/cm².

16. The transdermal delivery system of claim 15 wherein said predetermined amount of said fluid medium comprises about 3.0 mg/cm².

17. The transdermal delivery system of claim 3 wherein said first and second adhesive layers comprise adhesives which are at least partially resistant to plasticization by a solvent for said active agent.

18. A transdermal delivery system for delivering an active agent to the skin or mucosa of a patient comprising a backing layer, a polymer membrane disposed within said backing layer, an adhesive layer for attaching said transdermal delivery system to said skin or mucosa of said patient, a releasable layer for covering said adhesive layer prior to attachment of said transdermal delivery system to said skin or mucosa of said patient, and a fluid medium distributed between said adhesive layer and said polymer membrane in a manner such that between about 25% and 100% of said fluid medium is disposed in said polymer membrane.

19. The transdermal delivery system of claim 18 wherein between about 50% and 100% of said fluid medium is disposed in said polymer membrane.

20. The transdermal delivery system of claim 18 wherein a greater proportion of said fluid medium is disposed in said polymer membrane than is disposed in said adhesive layer.

21. The transdermal delivery system of claim 18 wherein said fluid medium comprises a liquid selected from the group consisting of a solvent for said active agent, an enhancer for said active agent, an excipient for said active agent and said active agent.

22. The transdermal delivery system of claim 18 wherein said adhesive layer comprises a first adhesive layer, and including a second adhesive layer disposed between said backing layer and said polymer membrane.

23. The transdermal delivery system of claim 18 wherein said polymer membrane has a capacity to retain greater than 5 mg/10 cm² of said fluid medium.

24. The transdermal delivery system of claim 23 wherein said fluid medium comprises a short chain alcohol.

25. The transdermal delivery system of claim 18 wherein said adhesive layer comprises a pressure-sensitive adhesive selected from the group consisting of acrylics, silicone, rubber, styrene-butadiene rubber, and polysisobutylene.

26. The transdermal delivery system of claim 25 wherein said pressure-sensitive adhesive comprises an acrylic-vinyl acetate resin.

27. The transdermal delivery system of claim 18 wherein said polymer membrane comprises a hydrophilic or hydrophobic polymer or copolymer.

28. A method for manufacturing a transdermal delivery system for delivering an active agent to a patient comprising
preparing an adhesive layer for attaching said transdermal delivery system to the skin or mucosa of said patient,
providing a polymer membrane,
impregnating said polymer membrane with a predetermined amount of a fluid medium for altering the rate of transmission of said active agent through said polymer membrane to said skin or mucosa of said patient,
drying said adhesive layer without drying said polymer membrane,
applying said adhesive layer to said impregnated polymer membrane,
providing a backing layer and incorporating said adhesive layer and said polymer membrane into said backing layer, and
providing a releasable liner adjacent to and protecting said adhesive layer prior to application of said transdermal delivery system to said skin or mucosa of said patient.

29. The method of claim 28 wherein said fluid medium comprises a liquid selected from the group consisting of a solvent for said active agent, an enhancer for said active agent, an excipient for said active agent, and said active agent.

30. The method of claim 28 wherein said adhesive layer comprises a first adhesive layer, and including applying a second adhesive layer between said backing layer and said polymer membrane.

31. The method of claim 30 wherein said first and second adhesive layers comprise an adhesive matrix including an adhesive selected from the group consisting of acrylic, silicone, polyisoalkalines, rubber, vinyl acetate, polyisobutylene rubber, polybutadiene, styrene-butadiene, cellulose derivatives, polysaccharides, polyurethane elastomers and polyester elastomers.

32. The method of claim 29 wherein said fluid medium comprises a solvent for said active agent.

33. The method of claim 32 wherein said solvent for said active agent comprises a C₂-C₁₈ alcohol.

34. The method of claim 31 including adding said active agent to said first adhesive layer.

35. The method of claim 31 including adding said active agent to said second adhesive layer.

36. The method of claim 31 including adding said active agent to said first and second adhesive layers.

37. The method of claim 28 wherein said polymer membrane comprises a polymer selected from the group consisting of hydrophilic and hydrophobic polymers and copolymers.

38. The method of claim 37 wherein said polymer is selected from the group consisting of polyolefins, ethylene-vinyl acetate, polyvinyl acetate, polyether block amides, polyurethane, polyamides, cellulose, cellulose derivatives, polyvinyl chloride, polyvinyl alcohol, polystyrene, polymethyl methacrylate, polysilane and polysiloxane.

39. The method of claim 28 wherein said predetermined amount of said liquid composition comprises from 0.5 to 10 mg/cm².

40. The method of claim 39 wherein said prededetermined amount of said liquid composition comprises from 1 to 7 mg/cm².

41. The method of claim 40 wherein said predetermined amount of said liquid composition comprises about 3.0 mg/cm².

42. The method of claim 30 wherein said first and second adhesive layers comprises adhesives which are at least partially resistant to plasticization by a solvent for said active agent.
